(19) United States
(12) Patent Application Publication
(10) Pub. No.: US 2008/0125666 A1
(43) Pub. Date: May 29, 2008

(54) Medical Monitoring System

(76) Inventors: Stuart Crozier, Queensland (AU); Stephen Wilson, Queensland (AU); Hang Ding, Queensland (AU)

Correspondence Address: NIXON & VANDERHYE, PC, 901 NORTH GLEBE ROAD, 11TH FLOOR, ARLINGTON, VA, 22203, US

(21) Appl. No.: 11/662,800

(22) PCT Filed: Jun. 10, 2005
(86) PCT No.: PCT/AU05/00839

§ 371 Date: Jun. 13, 2007

(30) Foreign Application Priority Data
Sep. 16, 2004 (AU) 2004905325

(51) Int. Cl. A61B 5/0402

(52) U.S. Cl. 600/509

(57) ABSTRACT

Biological data, such as human heart rate data, is acquired and processed in a non-linear manner to facilitate an assessment of the physiological state of the subject, and/or to assist in predicting incipient disorders or instability. Determinism, laminarity and recurrence measures are derived for a rolling sample of a time series of said data. The recurrence measure can be the Euclidean threshold (εₜₕᵣₑₛₕ) at a given recurrence rate. A representation, such a colour coded matrix or multi-dimensional vector, is formed from a combination of the derived determinism, laminarity and recurrence measures. The representation can then be analysed to detect indicators of physiological instability, such as arrhythmia, or to discriminate between arrhythmias. The analysis may be performed visually, or in an automated manner in real time, such as in an ambulatory or implanted device, or post hoc by a bedside monitor.

This invention relates to a medical monitoring system, and in particular, a system for predicting physiological arrhythmias. In a preferred embodiment, the invention comprises an ambulatory health monitoring and alarm system which utilises non-linear analysis of acquired electrocardiographic data in real time, and the generation of an alarm state or risk quantification for impending arrhythmia.

However, the scope of the invention is not necessarily limited thereto. Physiological time series data other than electrocardiographic signals can be subject to such analysis with the aim of predicting the likelihood of relevant system instability. Moreover, the invention may be embodied in ambulatory, implanted or fixed-bedside devices, as well as in post hoc analysis.

### BACKGROUND ART

[Mere reference to background art herein should not be construed as an admission that such art constitutes common general knowledge or prior art in relation to this application.]

Electrocardiographic (ECG) ambulatory monitoring systems are used to acquire signal for immediate analysis or post hoc analysis for the purpose of medical diagnosis, or the monitoring of medical management of cardiovascular disease whether by surgery, pharmaceutical or pacemaker means. Recording units typically acquire signal through a plurality of leads and electrodes applied to the subject, amplify and filter the acquired data, and store it in an analog fashion on magnetic tape, or in digitised form in an electronic storage medium.

Due to the limitations in memory size, it is commonplace to compress such data and consequently suffer loss in fidelity of that which is recorded. Analog recording systems require the replay of magnetic tape in order to view and analyse data retrospectively. This is time consuming and can also reduce the fidelity of the replayed data.

Analysis of recorded signals is largely limited to categorisation of abnormal beats or rhythm and measurement of their frequency during a period, typically 24 hours. Direct comparison techniques are used to diagnose these types of abnormalities. Average or instantaneous heart rates are used as primary measures for diagnosis.

It is recognised that the means by which heart rate is controlled cannot be adequately explained by control systems based on weighted linear combinations of physiological inputs. The non-linear behaviour of heart rate variability has been recognised as exhibiting chaotic features recognisable through mathematical techniques developed for such systems. Furthermore, the absolute value of chaotic parameters or changes therein can be markers of illness or dynamic state changes related to, or predisposing one to, illness.

There exist specific algorithms or methods of analysis, which are based on the non-linear behaviour of the derived signal. The chaotic nature of a time series signal can be characterised by a suite of measures with applicability to the clinical state of the subject. Well controlled acquisition of heart rate data has led to the acceptance of such measures in medical disciplines, in particular the field of cardiology.

A shortcoming of many methods of non-linear analysis is the susceptibility of the technique to noise (of any source) and non-stationarity of the dynamic control. The term “non-stationarity” refers to the change of control state over the period of data capture. If the “rules” governing heart rate regulation change, then such methods used for analysis of the signal are flawed. One recent technique, which combats this shortcoming, is a method of recurrence analysis¹ based upon the embedding of time series data, and a multi-dimensional vector is then used to represent the control state of the dynamic system (such as heart rate regulation) as a vector quantity in multi-dimensional space. The predictive value of the recurrence plot in isolation has been acknowledged and described by others². The predictive value of another non-linear technique, specifically, using Poincare plots of the cardiotachogram has also been disclosed³. Beat to beat interval time series is the primary data source but the multidimensional embedding process is not performed in this technique.

The fundamental mathematical theory underling Recurrence Qualification Analysis (RQA) has been disclosed⁴. If the experimental data series are (x(1), x(2), x(3), x(4), x(M)}, the recurrence plot (RP) can be expressed as an array in a N×N dimension

R(i,j)=Θ(ε−|Y(i)−Y(j)|) (1)

where ε is the normalised Euclidean threshold; Y is the phase space vector and Θ is the Heaviside function.

### The two components in the delay-embedding construction in phase space are Y(i) and Y(j), which can be mathematically expressed as;

Y(i)={x(i), x(i−τ), . . . , x(i−(dE−1).τ)} (2)

Y(j)={x(j), x(j−τ), . . . , x(j−(dE−1).τ)} (3)

where τ is the “lag” parameter.

An additional parameter which may be derived from the RP is defined as the Euclidean threshold at a given recurrence rate (REC εₜₕᵣₑₛₕ₎). This value is a measure of the minimal Euclidean distance at which E must be set to achieve a prescribed recurrence rate. It can be seen that the recurrence is a function of the chosen ε as per equation 4 below

REC=f(ε) (4)

It can be shown that the inverse of the above equation cannot be found due to the undefined dynamic behaviour of the data. In order to find the minimal ε which will generate a given REC, a numerical solution must be employed. The monotonic relationship between REC and ε permit the use of the bisection method whereby an initial “seed” value for ε is applied to the data using the RP and resulting REC observed. Subsequent ε values are the bisection of the distance between current value and the boundary of the interval over which the search is performed.

It is found that this embedded vector representing the dynamic behaviour of the physiology migrates over time, but revisits regions of this space. Should such recurrences or revisitations occur in a consecutive sequential fashion, it is indicative of rule obeying dynamic control being expressed in the time series. This behaviour can be objectively quantified from the recurrence matrix and used as a marker of health or illness expressed through physiological control. Studies performed on defined cardiac and respiratory illness have demonstrated the benefit of recurrence analysis in revealing behaviour not seen in conventional analysis.

Specifically, the measure of determinism or rule obeying behaviour can indicate the physiological state of the subject based upon beat-to-beat variability of heart rate or breathing rate. RP provides measurable parameters concerning the properties of a deterministic chaotic system. One of its advantages as an analysis tool is that it does not require long experimental data series to capture chaotic properties. Based on the recurrence plot (RP), recurrence qualification analysis (RQA) was developed as a tool to measure these chaotic properties quantitatively. It has been observed that RP appears to “mirror” the beat to beat interval changes by the recurrences. It has also been found that determinism changes reflect the different physiological stages of an experimental heart rate observation experiment.

Recurrence plots have been applied to the quantification of various physiological parameters such as respiration⁵ or muscle activity derived from electromyographic signals (EMG)⁶. It is the common conclusion from such work that changes in the control system dynamics often precede any observation of system change seen in the simple time series data.

In the RQA method, determinism (DET), laminarity (LAM) and recurrence (REC) represent three important dynamic properties. REC, or recurrence rate, is the density of recurrence points and quantifies the percentage of recurring points in the RP. A recurrence point implies that the dynamic state difference of two points falls within a relatively low range (Euclidean threshold) in phase space. For a chaotic system, when the dynamic is visiting a region of an attractor, its dynamic behaviour follows a certain pattern and maintains a similar pattern when revisiting the same region of the attractor. This kind of revisiting normally results in a diagonal line in the RP.

DET (the percentage of the recurrence points forming the diagonal line points) represents the frequency of repetition of certain patterns in the experimental series. Vertical and horizontal lines result when a relatively “quiet” section or laminar state (LAM) in the experimental series exists, and are quantified in a similar fashion to determinism. Observations of RPs derived from heart rate variability series reveal that the DET, LAM and REC are closely correlated to each other.

A refined measure of REC is the derivation of the Euclidean threshold (εₜₕᵣₑₛₕ) at a given recurrence rate. This value represents the minimal distance criterion used to judge the co-occurrence or recurrence of vectors in high dimensional space. εₜₕᵣₑₛₕ is thereby a value, reflecting the proximity of the vectors Y(i) and Y(j) in space. It will have the units of the inverse period of the data (beats per minute).

When the heart rate control system transits from one state to another, i.e. from resting to exercising, the DET, LAM and REC will vary corresponding to the transition. In some cases, this transition follows a pattern and the same pattern repeats when a similar transition reoccurs. The physiological meaning of DET and LAM may vary due to the variation of the REC. For example, if the REC in a local area is elevated, an elevated DET and LAM will be found, but the significance of these values (DET and LAM alone) is questionable.

The rich structures in the RP contain more information than the averaged values of DET, LAM and REC when viewed over the entire RP.

Such a method as recurrence quantification can be implemented on a personal computer for analysis of signals in a post hoc fashion. However, the nature of the calculations and memory requirement preclude the use of such a technique in an ambulatory or implantable device.

It is an aim of this invention to combine the DET, LAM and REC properties of recurrence plots in a new manner, to give advantageous diagnostic and predictive indicators.

It is a preferred aim of this invention to provide a medical monitoring system which incorporates such a technique in an ambulatory, fixed or implanted device and performs recurrence analysis in a real time fashion. Such a technique will thereby permit an alarm or alert function to be implemented with benefit for both subjects and clinicians

### SUMMARY OF THE INVENTION

In a broad form, the invention provides a method of processing or analysing biological data acquired from a subject to assess the physiological state of the subject, and/or to assist in predicting incipient disorders or instability in the short or long term, the method comprising the steps of:

obtaining a time series of said data from the subject;

deriving determinism, laminarity and recurrence measures for a rolling sample of said data;

forming a representation of a combination of the derived determinism, laminarity and recurrence measures; and

analysing the representation to detect indicators of instability in the physiological state of the subject.

Preferably, the recurrence measure is the Euclidean threshold (εₜₕᵣₑₛₕ) at a given recurrence rate.

The rolling sample is a moving “window” or meta-window of data. This enables the technique to be applied in real time.

The deriving step includes forming a recurrence plot, from which determinism, laminarity and recurrence are derived. By using a combination of the derived determinism, laminarity and recurrence measures, a more reliable indication of likely instability is obtained.

Preferably, the determinism, laminarity and recurrence measures are combined in a colour-encoded matrix, to facilitate its analysis. However, other representations of the combined determinism, laminarity and recurrence measures, such as the εₜₕᵣₑₛₕ may be employed.

The analysing step may be performed manually, i.e. visually, or by suitable pattern recognition software, to detect patterns and/or colours indicative of incipient instability in the physiological state of the subject.

Typically, the analytical technique of this invention is applied to heart rate data obtained using a single lead surface electrocardiogram (ECG). However, although the primary data series used by way of example in this invention is heart beat-to-beat interval (cardio-tachogram), the invention is not limited to human cardiac signal analysis. The technique can be applied to other physiological signals.

Preferably, the invention is embodied in an ambulatory device, such as a Holter type monitor. Alternatively, the invention can be embodied in a stationary (bedside) monitor system. In one particular application, the technique of the invention is integrated into the function of an implantable cardioversion device (ICD). The ICD can deliver a direct current defibrillation shock responsive to the outcome of the method described above.

In another form, the invention provides apparatus for processing biological data acquired from a subject to assess the physiological state of the subject, and/or to assist in predicting incipient disorders or instability in the short or long term, comprising sensing means for obtaining a time series of said data from the subject;

processing means for deriving determinism, laminarity and recurrence measures for a rolling sample of said data; and

means for forming a representation of a combination of the derived determinism, laminarity and recurrence measures, for analysis.

The apparatus may also include means for automated analysis of the representation of the combined determinism, laminarity and recurrence measures, and alarm means responsive to the analysis means for signalling an alarm condition upon detection in the representation of an indication of incipient instability in the physiological state of the subject.

The apparatus may be embodied in an ambulatory device, such as a Holter type monitor. Alternatively, the invention can be embodied in a stationary (bedside) monitor system, or an implantable cardioversion device (ICD).

This invention is therefore based on the recognition that non-linear analysis, and in particular, a combination of determinism, laminarity and recurrence measures, is a better descriptor of the behaviour of cardiovascular control and has predictive capabilities with respect to dangerous arrhythmias and/or asystole. The invention enables the implementation of such analyses in real time or in post hoc analysis.

In order that the invention may be more readily understood and put into practice, one or more preferred embodiments thereof will now be described, by way of example only, with reference to the accompanying drawings.

### BRIEF DESCRIPTION OF THE DRAWINGS

FIG. 1 is a perspective view of an ambulatory monitor according to one embodiment of the invention. A casing (60×12×40 mm) B Electrodes for application to subject C Slot for memory card, MMC or similar. E graphic display for setup and signal review.

FIG. 2 (a) is a conventional recurrence plot (RP) of a human tachogram commencing in sinus rhythm and progressing to the rapid rate of ventricular tachycardia.

FIG. 2 (b) is a DLR recurrence plot and cardiotachogram of a human subject displaying atrial fibrillation throughout the duration of the series. The relative density of deterministic structures characterises this pattern.

FIG. 2 (c) is a DLR plot of a human subject with multiple episodes of supra-ventricular tachycardia. A “wandering” pattern is seen as the dynamic state migrates from sinus rhythm into a rapid disordered pattern and returns.

FIG. 2 (d) is the DLR plot of a data series prior to and including ventricular tachycardia. The dense laminarity and deterministic pattern is seen immediately prior to the onset of the arrhythmia. Sinus rhythm is presented by the flat cardiotachogram in region A. Increase in yellow colour banding is seen in B with intense LAM and DET seen at C immediately prior to ventricular tachycardia at D.

FIG. 3 represents the pixel summation of predefined colour bands from a DLR plot over time. The data is derived from the DLR plot of FIG. 2 (c). The duration of threshold crossing and lead time prior to the onset of arrhythmia are indicated. Crossings of the threshold of short duration as seen here are not viewed as significant events

FIG. 4 Shows the time series plot of a 100 beat-beat meta-window analysis of determinism, laminarity and REC εₜₕᵣₑₛₕ. The point at which a lower 95% confidence interval is crossed is indicated by arrow. This point occurs during sinus rhythm and is some 140 beats or 3 minutes prior to the arrhythmia. Onset of ventricular tachycardia is at the end of the series.

FIG. 5 is a flowchart of a DLR algorithm as applied to continuous analysis of heart rate variability and detection of dynamic state changes prior to arrhythmia. The colour and density of pixels in the DLR plot are the form the basis on which an alarm status is generated.

FIG. 6 is a flowchart describing the steps in the determination of likely arrhythmia. The RECεₜₕᵣₑₛₕ, DET and LAM are continually updated by a moving meta-window and changes from the normal distribution of such parameters used as the basis for discrimination and alarm status.

### DESCRIPTION OF PREFERRED EMBODIMENT(S)

The basis of the method of the preferred embodiment of the invention commences with the construction of a recurrence matrix or recurrence plot (RP). To enable maximum information to be derived from the RP, a new 2 dimensional derivative matrix of the RP is used. This matrix is constructed on the basis of the individual values for determinism, laminarity and recurrence (DLR) at every point in the existing RP. The structure and colour of this “DLR” matrix can be interpreted to reveal indicative physiological state changes.

The construction of the DLR is governed by the equations below.

The DLR RP is actually the distribution of the two dimensional trends of DET, LAM and RR, which are presented by combined colours. The two dimensional trends DLR(i,j) can be expressed as:

$$\begin{matrix}DLR ( i , j ) = RGB ( RD ( i , j ) , GR ( i , j ) , BL ( i , j ) ) . \\ where \text{:} & ( 5 ) \\ RD ( i , j ) = 0 \times FF \cdot {DET}_{2 D} ( p , q ) & ( 6 ) \\ GR ( i , j ) = 0 \times FF \cdot {LAM}_{2 D} ( p , q ) & ( 7 ) \\ BL ( i , j ) = 0 \times FF \cdot {RR}_{2 D} ( p , q ) \\ and & ( 8 ) \\ {LAM}_{2 D} ( p , q ) = \frac{{\sum }_{i , j = p , q}^{P + {N}_{wnd} , q + {N}_{wnd}} {R}_{LAM} ( i , j )}{{\sum }_{i , j = p , q}^{P + {N}_{wnd} , q + {N}_{wnd}} R ( i , j )} & ( 9 ) \\ {DET}_{2 D} ( p , q ) = \frac{{\sum }_{i , j = p , q}^{P + {N}_{wnd} , q + {N}_{wnd}} {R}_{DET} ( i , j )}{{\sum }_{i , j = p , q}^{P + {N}_{wnd} , q + {N}_{wnd}} R ( i , j )} & ( 10 ) \\ {RR}_{2 D} ( p , q ) = \frac{{\sum }_{i , j = p , q}^{P + {N}_{wnd} , q + {N}_{wnd}} R ( i , j )}{{N}_{wnd}^{2}} & ( 11 )\end{matrix}$$

where
R_{DET}: DET Bitmap image. If R(i,j) and its neighbours can form a diagonal line which meets diagonal line criterion of >=Sₘᵢₙ, the value of R_{DET}(i,j) is 1. Otherwise R_{DET}(i,j)=0.
R_{LAM}: LAM Bitmap image. If R(i,j) associating its neighbours can form a vertical line VL or horizontal line HL which meets the length criterion of VL>=Vₘᵢₙ, or the length of HL>=Wₘᵢₙ, the value of R_{DET}(i,j) is 1. Otherwise R_{LAM}(i,j)=0.
N_{Wnd}: meta window size.
Vₘᵢₙ: Minimum vertical line length.
Wₘᵢₙ: Minimum horizontal line length.

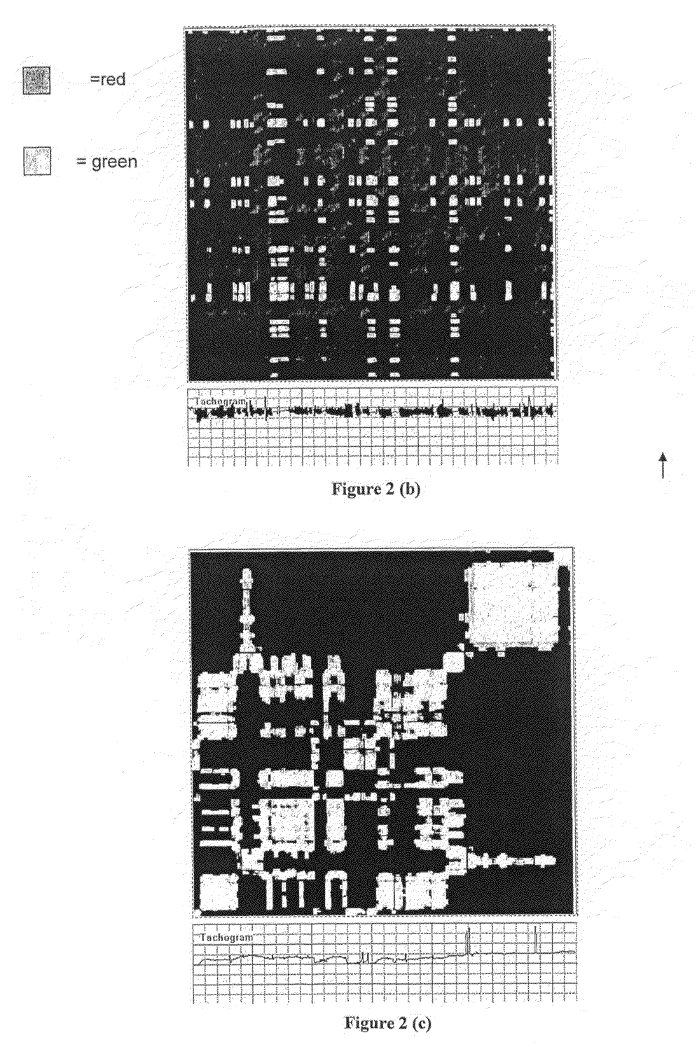

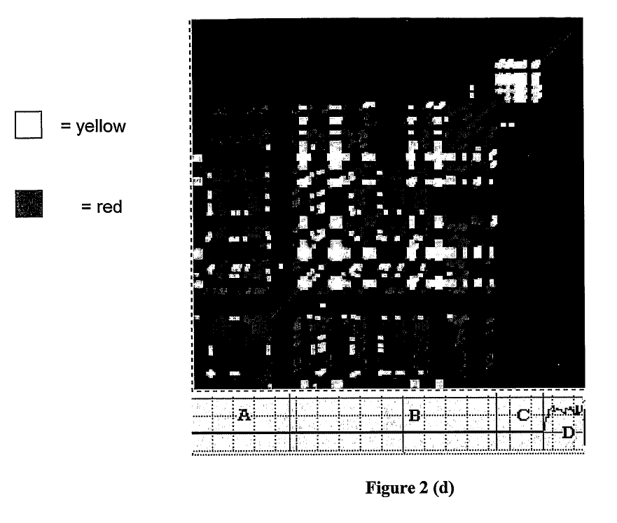

By extracting a specific colour, certain dynamic behaviour can be extracted in terms of the densities of the RP dots, recurrences and laminar states. The two colours extracted in FIG. 2(d) can be used to represent two different dynamic behaviours. The yellow areas represent the high density of recurrences with mixed laminar states, which, in this case, is generated by the process of consecutively revisiting a same region of phase space. The two similar yellow areas in the cross sections (left up; right down) implies that the two processes being trapped are identical in terms of duration, region and the frequency of revisit.

An RP derived from a subject progressing from normal sinus rhythm into ventricular tachycardia (VT) is shown in FIG. 2. FIG. 2(a) shows a conventional RP and tachogram below. FIG. 2(d) shows a DLR plot of the same series. This graphic provides a clear view of the distributions and densities of recurrences and laminar states.

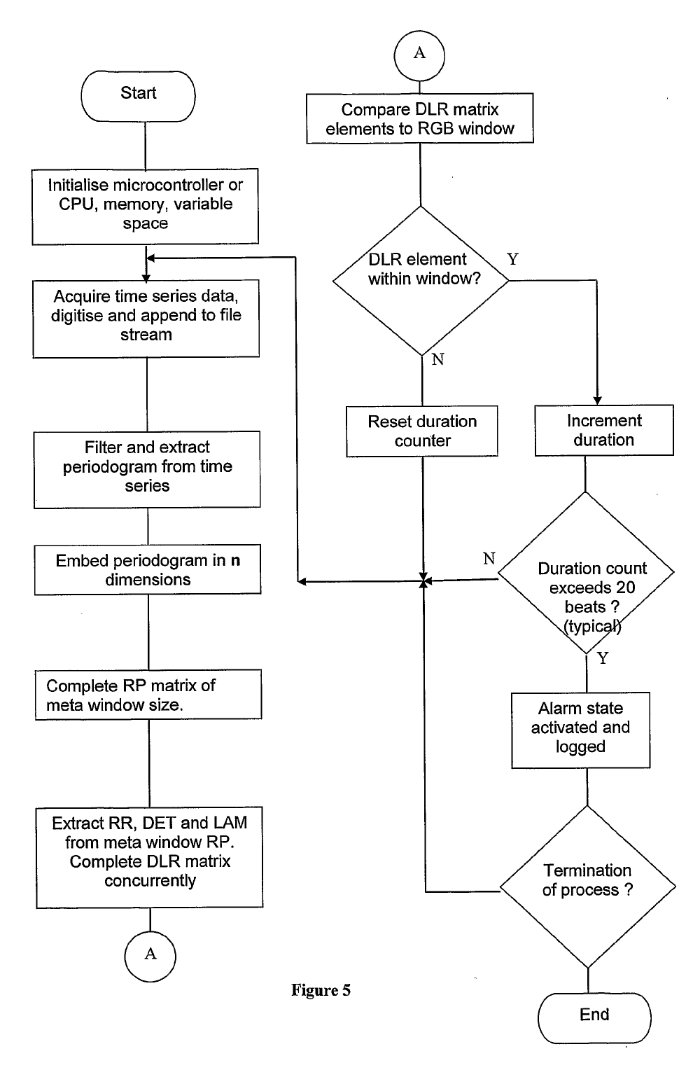

In a first embodiment, a fixed system in which biosignals representative of heart rate are digitised, stored and analysed using the DLR method is implemented as per the flowchart of FIG. 5. A bioamplifier provides signal conditioning to biopotentials obtained from surface electrodes applied to the subject. An analog to digital conversion provides a raw signal from which a beat-to-beat interval can be found using known techniques. This period versus time, or tachogram, is then a suitable data stream for application to the DLR method. Such a fixed system has application, for example, to bedside monitoring for the purpose of real time alarm activation. Analysis of data after it has been collected is of use for identifying at risk patterns. Therapeutic actions may then be taken on the basis of this analysis.

A second embodiment is optimised for ambulatory or portable use. The purpose of such a device is primarily for alarming the subject and/or clinician of the incipient risk of potentially dangerous rhythms. A storage function allows post hoc analysis and archived alarm states to be retrieved for review and the exercising of therapeutic options.

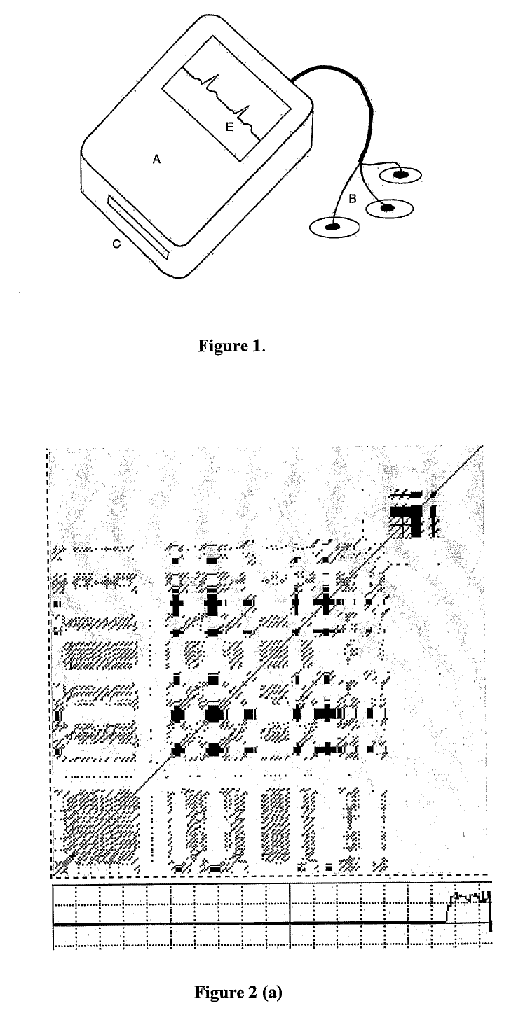

The ambulatory recording device is formed in the shape shown in FIG. 1 with a display window and a plurality of user operated switches. A cable consisting of a number of conductive leads exits the enclosure and is applied to the subject. A removable memory card is accessible but hidden for normal use.

The display can show real time signal as well as confirm operating status to the user.

The device can be operated by firmware to carry out the two general roles of managing an operating system and recording, as well as analysis in which an implementation of recurrence analysis is operating.

A signal acquired from a periodic biosignal such as heartbeat or pneumogram is differentiated and compared to a threshold to produce a signal in synchrony with the normal heart beat or similar physiological variable. The interval between these events is the primary data source for application to the recurrence algorithm. Embedding of this signal is performed by the creation of a kernel consisting of an array of m samples. The choice of the value of m is governed by a general relationship:

m=2n+1 (11).

where n is the number of governing inputs influencing the dynamic controller. In a typical case this may be 6. Empirically it may found that disease states are characterised adequately by low-dimensional dynamics. In this case an embedding dimension of 2 or 3 may be used with success.

The kernel of m samples is updated with the acquisition of each subsequent beat-to-beat interval. The vector produced from this data set is compared with previous vectors to predefined period back in time. The duration of data used for this determination is based on the difference in relative frequency of state changes due to natural controller migration and the onset of potentially dangerous rhythms. This value is determined in an empirical fashion. A moving window of the data is thus recurrence tested and the derived measures of recurrence, determinism and laminarity recorded.

In this manner, the requirement for a massive memory space in which to analyse a 24 hour heart rate record is avoided. Such a technique can now be implemented in an embedded processor and housed in a physical form suitable for an ambulatory monitor.

A string of recurrences will represent the dynamic system following a rule for the period of such a string of values. It is known that such behaviour can be the basis of a diagnostic process. FIG. 2 (a) illustrates the progression of recurrences forming a deterministic feature. Such events may be the basis of initiating an alarm or raising the awareness state of the subject or clinician.

The DLR matrix resultant from analysis of heart rate recordings can be seen to contain patterns and texture qualities which signify dynamic changes prior to the onset and during the occurrence of a ventricular tachycardia. FIG. 2(d) shows the pattern changes from a typical sample of beat to beat intervals. The tachogram present as a time series shows the point at which a malignant rhythm commenced. The onset of the arrhythmia is seen to lag the observable pattern changes in the matrix above. Quantification of this observation is possible using known descriptors and techniques in the field of pattern matching or shape detection. Such morphometric techniques may be optimised for differing rhythm disturbances.

The patterns within the DLR matrix as illustrated in FIGS. 2 (b), (c) and (d) may be recognised or interpreted visually, but may be optimally recognised or interpreted using an automated or semi-automated mathematical technique. The properties of the changes prior to instability are characterised by textural and pattern changes in the DLR matrix. Such qualities are well recognised in the field of machine vision and can be quantified using known techniques. Although there exists no formal definition of texture 7 there exist many techniques which can determine the difference in texture or frequency of variation between images or regions of an image^{8 9 10}. In addition, methods generating a spatial dependence matrix or co-occurrence matrix can quantify the spatial autocorrelation properties of an image. Measures of entropy and linearity can be extracted from such an analysis, which are measures of textural content¹¹. Such automated pattern recognition techniques can be applied to the interpretation of the colour matrix generated by the method and apparatus of this invention, and the subject matter of the references listed in the appendix hereto is incorporated herein by reference.

Patterns observed in the DLR matrix can be used as a basis for discrimination between arrhythmias. The patterning of the dynamic control of heart rate is then a possible diagnostic feature. FIGS. 2 b, c and d illustrate the typical patterns seen in arrhythmias of differing origins. Atrial fibrillation, supraventricular arrhythmia and ventricular tachycardia are given as examples due to the differing anatomical and electrophysiological basis of each rhythm disturbance.

Some of the pattern recognition techniques referred to above may not be ideally suited to the implementation of this invention in a portable or ambulatory device due to processing and memory constraints. A simpler technique not based on pattern recognition is described below and is used in an analysis of sixteen cardiotachograms from which ventricular tachycardia ensues.

To demonstrate the efficacy of the DLR method, periods which generate a DLR pixel in a specified RGB window, are detected. Such a technique can thereby detect dynamic behaviour typified by any combination of determinism, laminarity or recurrence. A meta-window of typically 100 beats is examined as it moves along the time series. A determination of Euclidean threshold (Eₜₕᵣₑₛₕ) is performed for a given recurrence rate. A value of 10% is an appropriate value for this rate as it reflects local recurrences⁶. εₜₕᵣₑₛₕ is seeded with a finite value and successive approximations made until the recurrence rate of 10+/−1% is obtained. This minimal Euclidean distance is then the representation of the recurrence behaviour for that 100 value window. Laminarity and determinism estimates from the 100 beat window are also performed. The combination of determinism, laminarity and recurrence behaviour as expressed by its Euclidean threshold can be used as a discriminator for the purpose of detecting heart rate dynamics associated with arrhythmia.

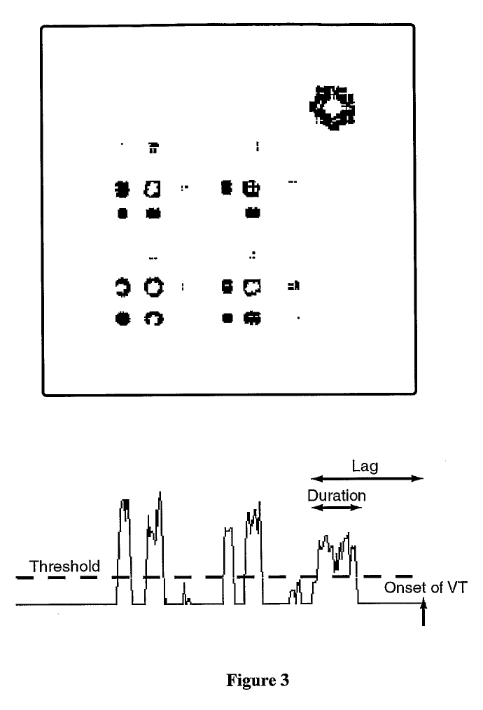

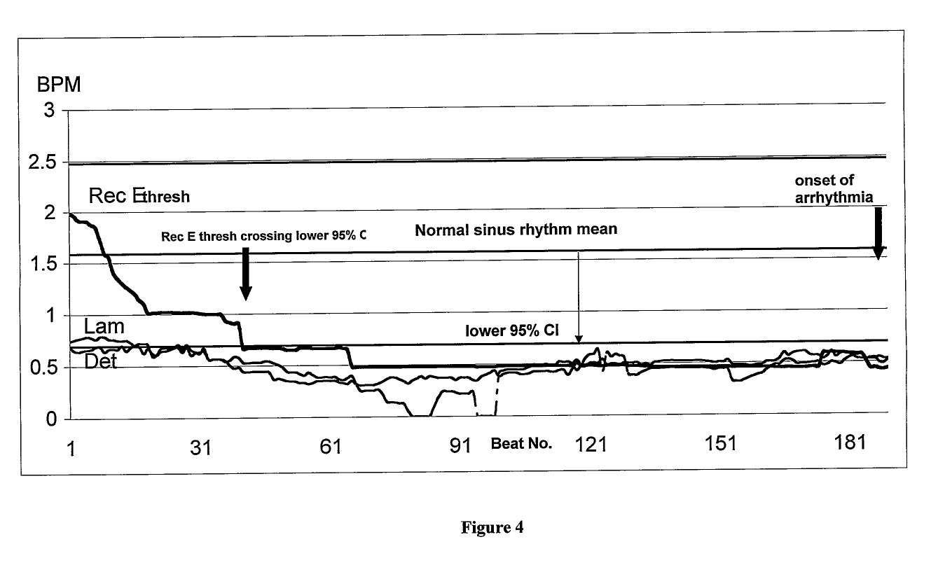

FIG. 4 illustrates the evolution of determinism, laminarity and εₜₕᵣₑₛₕ, during sinus rhythm prior to the onset of ventricular tachycardia. The mean and 95% confidence intervals for εₜₕᵣₑₛₕ are also illustrated. The crossing of the lower 95% confidence interval of the normal distribution is a possible defining point in time after which the arrhythmia may be deemed likely. The flowchart of FIG. 5 illustrates one example of such a process used to test the predictive properties of the DLR method. A sample of 16 time series derived from different human subjects prior to onset of ventricular tachycardia is presented in table 1. Patterns from the DLR matrix expressing the patterns outlined in FIG. 3 (c) were detected. Each tachogram contains sinus rhythm prior to the onset of the tachycardic episode.

By means of this summary result, it can be seen that such a non-linear technique can exhibit prediction or generate a likelihood of ensuing instability at varying durations prior to the event. Although the primary data series used for this illustration is derived from heart rate, it is entirely possible that other physiological time series can be applied in a similar fashion with similar predictive properties. Alarm states or clinical action can then occur on the basis of such a result. The algorithm may be implemented in a post hoc fashion or real-time in an ambulatory device or fixed monitor. It is well known that implanted cardiac devices such as pacemakers and implantable cardioversion devices contain hardware for recording and analysing heart rate and breathing rate signals. The programmable nature of such devices would permit the embodiment of the algorithms described herein for the purpose of generating alarm states and exercising therapeutic actions such as pacing and/or defibrillation pulses.

The foregoing embodiments are illustrative only of the principles of the invention, and various modifications and changes will readily occur to those skilled in the art. The invention is capable of being practiced and carried out in various ways and in other embodiments. It is also to be understood that the terminology employed herein is for the purpose of description and should not be regarded as limiting.

### Results

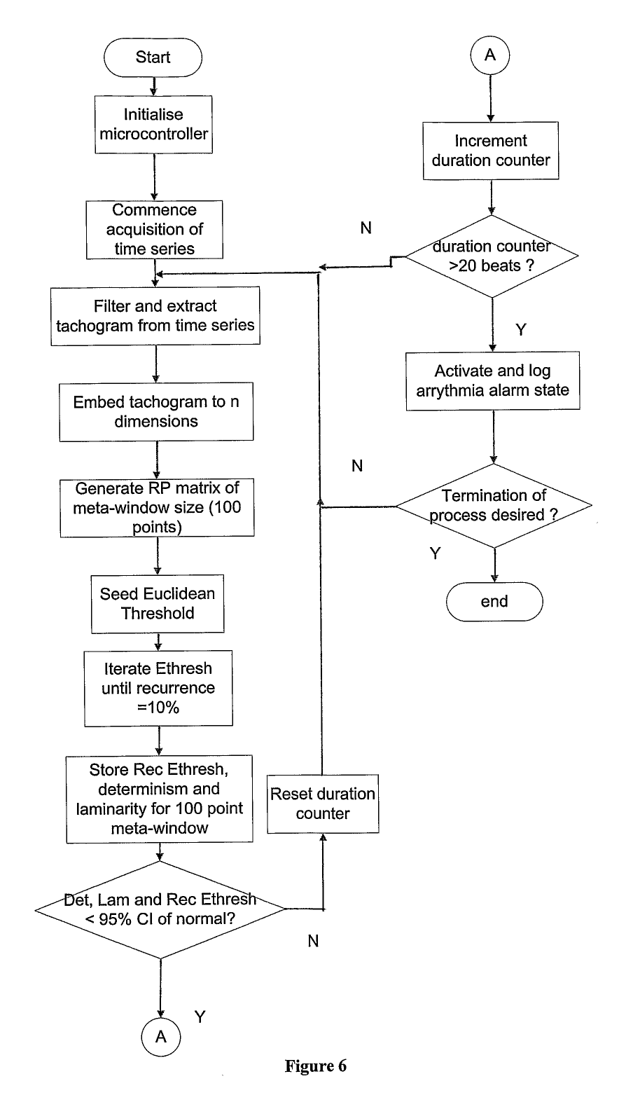

Analysis of 6, records of sinus rhythm progressing to ventricular tachycardia are summarised in table 1 below. The method of analysis is based on the DLR plot and algorithm described in FIG. 6 and represented by the result seen in FIG. 4. It can be seen that threshold crossings occur prior to arrhythmia onset and occur at variable times. It can be inferred that optimum settings of the threshold and colour may change the behaviour of this tool as a discriminator between normal and disease states.

TABLE 1

| | | Mean duration of | |
|---|---|---|---|
| Case | No. of threshold | threshold | Lag to onset of |
| No. | crossings | crossing (beats) | tachycardia (beats) |
| 1 | 1 | 7 | 105 |
| 2 | 1 | 20 | 56 |
| 3 | 1 | 30 | 156 |
| 4 | 3 | 31 | 26 |
| 5 | 4 | 25 | 45 |
| 6 | 1 | 15 | 110 |

### Summary analysis is presented of 16 individual adult human cardiac tachogram records prior to onset of ventricular tachycardia using the numerical technique outlined herein and shown graphically in FIGS. 4 and 6.

### A meta-window of 100 beats up to the onset of ventricular tachyeardia was used to calculate the REC εₜₕᵣₑₛₕ, the DET and LAM.

Mean REC εₜₕᵣₑₛₕ and standard deviation for normal sinus rhythm data of equal time duration from 10 subjects of similar age distribution analysed on the basis of a 10% recurrence rate, Wₘᵢₙ=4 and Vₘᵢₙ=4 were 2.0 (6.9) BPM. DET and LAM were 20% (5.9) and 19% (1.5) respectively. The mean of the 16 equivalent values for analysis prior to ventricular tachycardia were REC εₜₕᵣₑₛₕ 3 BPM, DET 46% and LAM 65%. The difference between distributions of the 16 VT subjects were significantly different (p<0.01) to the mean values from normal sinus rhythm data (t>3.05). This result shows the possibility of discrimination between normal beat-beat variation and beat-beat variation present prior to the onset of a potentially hazardous arrhythmia.

### REFERENCES

¹ Eckmann, J, P. Kamphorst, S. O. Ruelle, D. Recurrence plots of dynamical systems Europhysics Letters 5, 973, 1987.
² Marwan, N. Wessel, N. Meyerfelt, U. Schirdewan, A. Kurths, J. Recurrence-plot-based measures of complexity and their application to heart-rate variability data Physical Review E 66, 026702. 2002
³ Levitan J. Lewkowicz, M. Method and system for measuring heart rate variability U.S. Pat. No. 6,731,974 B2 May 4, 2004.
⁴ Slovut, D. P. Bolman, R. M. Bianco R. W. Non-invasive detection of rejection in heart transplant patients. U.S. Pat. No. 5,285,793
⁵ Wilson, S. J, Suresh, S, Williams, G. Harris, M. Cooper, D. Analysis of infant respiratory dynamics using recurrence plot strategies Proc. Am. Thoracic Society Scientific Meeting. Orlando, Fla. May 2004.
⁶ Webber, C. Zbilut, J. Analysis of physiological systems using recurrence quantification techniques. J. Appl. Physiol. 76, 965. 1994.
⁷ Gonzalez, R. C. and Woods, R. C. (1992) Digital Image Processing, Reading, Mass.: Addison-Wesley.
⁸ Haralick, R. M. (1979) Statistical and structural approaches to texture, IEEE Transactions on Systems, Man and Cybernetics, 4(7), pp. 394-396.
⁹ Lu, S. Y. and Fu, K. S. (1978) A syntactic approach to texture analysis, Computer Graphics and Image Processing, 7(3), pp. 303-330
¹⁰ Tomita, F., Shirai, Y., Tsuji, S. (1982) Description of texture by structural analysis, IEEE Transactions on Pattern Analysis and Machine Intelligence, 4(2), pp. 183-191.
¹¹ Weszka, J, Dyer, C., Rosenfeld, A. (1976) A comparative study of texture measures for terrain classification, IEEE Transactions on Systems, Man and Cybernetics, 6(4), pp. 269-285.

1. A method of processing biological data acquired from a subject to assess the physiological state of the subject, and/or to assist in predicting incipient disorders or instability in the short or long term, the method comprising the steps of:
obtaining a time series of said data from the subject;
deriving determinism, laminarity and recurrence measures for a rolling sample of said data;
forming a representation of a combination of the derived determinism, laminarity and recurrence measures; and
analysing the representation to detect indicators of instability in the physiological state of the subject.

2. A method as claimed in claim 1, wherein the recurrence measure is the Euclidean threshold (εₜₕᵣₑₛₕ) at a given recurrence rate.

3. A method as claimed in claim 1, wherein the deriving step includes forming a recurrence plot, from which the determinism, laminarity and recurrence measures are derived.

4. A method as claimed in claim 1, wherein the representation is a colour-encoded matrix.

5. A method as claimed in claim 1, wherein the representation is a three-dimensional vector.

6. A method as claimed in claim 1, wherein the analysing step comprises detection of patterns and/or colours in the representation indicative of incipient instability in the physiological state of the subject.

7. A method as claimed in claim 6, wherein the biological data is human heart rate data obtained using a electrocardiogram (ECG), and the detection comprises qualitatively identifying arrhythmias based on visualisation of the representation.

8. A method as claimed in claim 6, wherein the biological data is human heart rate data obtained using a electrocardiogram (ECG), and the analysing step includes post hoc analysis to discriminate between arrhythmias and/or to assess relative risk of arrhythmias.

9. A method as claimed in claim 1, wherein the method is carried out in an ambulatory device.

10. A method as claimed in claim 1, wherein the analysing step includes detecting the presence of a predetermined value at one of more predetermined locations in the representation.

11. Apparatus for processing biological data acquired from a subject to assess the physiological state of the subject, and/or to assist in predicting incipient disorders or instability in the short or long term, comprising
sensor for obtaining a time series of said data from the subject; and
processor in data communication with the sensor for
(i) deriving determinism, laminarity and recurrence measures for a rolling sample of said data; and
(ii) forming a representation of a combination of the derived determinism, laminarity and recurrence measures, for analysis.

12. Apparatus as claimed in claim 11, wherein the recurrence measure is the Euclidean threshold (εₜₕᵣₑₛₕ) at a given recurrence rate.

13. Apparatus as claimed in claim 11, wherein the apparatus is embodied as an ambulatory device.

14. Apparatus as claimed in claim 11, wherein the apparatus is embodied in a device implantable in the human body.

15. Apparatus as claimed in claim 11, further comprising a display, and wherein the representation is a colour-encoded matrix output to the display for qualitative analysis.

16. Apparatus as claimed in claim 11, further comprising an analysing device for automated analysis of the representation.

17. Apparatus as claimed in claim 16, further comprising an alarm responsive to the analysing device for signalling an alarm condition upon detection in the representation of an indication of incipient instability in the physiological state of the subject.

18. Apparatus as claimed in claim 11, wherein the biological data is human heart rate data, and the sensing means includes ECG electrodes for obtaining the data.
